(19) United States
(12) Patent Application Publication
(10) Pub. No.: US 2013/0022577 A1
(43) Pub. Date: Jan. 24, 2013

(54) PROBIOTIC COMPOSITIONS USEFUL FOR TREATMENT OF BIPOLAR DISORDER

(71) Applicants: Mark L. COBB, Dallas, TX (US); Alyson J. Cobb, Dallas, TX (US)

(72) Inventors: Mark L. COBB, Dallas, TX (US); Alyson J. Cobb, Dallas, TX (US)

(73) Assignee: Cobb & Associates, Dallas, TX (US)

(21) Appl. No.: 13/591,217

(22) Filed: Aug. 21, 2012

Related U.S. Application Data
(63) Continuation-in-part of application No. 11/535,752, filed on Sep. 27, 2006, now Pat. No. 8,246,946.
(60) Provisional application No. 60/720,869, filed on Sep. 27, 2005.

(51) Int. Cl. A61K 35/74; A61P 25/00

(52) U.S. Cl. 424/93.3

(57) ABSTRACT

Compositions and methods for treatment or prophylaxis of a bipolar disorder disease state, in which the compositions include the bacilli (1) Bacillus subtilis, (2) Bacillus coagulans, and (3) Enterococcus faecium. The compositions may be embodied in suitable dose forms such as capsules containing the microbial species in combination with a substrate for the microbial species, e.g., fructo-oligosaccharides, for oral administration according to a predetermined dosage schedule.

### CROSS-REFERENCE TO RELATED APPLICATIONS

This is a continuation-in-part under 35 USC 120 of U.S. patent application Ser. No. 11/535,752 filed Sep. 27, 2006 in the names of Mark L. Cobb and Alyson J. Cobb for TREATMENT OF BIPOLAR DISORDER UTILIZING ANTI-FUNGAL COMPOSITIONS. Aug. 21, 2012 issued as U.S. Pat. No. 8,246,946, which in turn claims the benefit under 35 USC 119 of U.S. Provisional Patent Application 60/720,869 filed Sep. 27, 2005 in the names of Mark L. Cobb and Alyson J. Cobb. The disclosures of said U.S. patent application Ser. No. 11/535,752 and said U.S. Provisional Patent Application 60/720,869 are hereby incorporated herein in their respective entireties, for all purposes.

### BACKGROUND OF THE INVENTION

The present invention in a broad aspect relates to a probiotic composition for treatment of bipolar disorder, and to a method for treating a patient suffering from such disease state, involving administration of the probiotic composition to such patient.

### DESCRIPTION OF THE RELATED ART

A variety of disease states and adverse physiological conditions have an etiological relationship to fungal exposure and infection. For example, candidiasis is a common fungal infection of mucosal membranes and other tissues. The infection is caused by the yeast-like organism Candida. Numerous species of Candida exist, including C. albicans. The recent increase in candidiasis is most likely caused by the rising incidence of AIDS, more intensive regimens of cancer therapy, complications of abdominal or cardio-thoracic surgery, organ transplantations, burns and trauma. In addition, immunocompromised individuals and women of childbearing age, especially pregnant women or women with one or more childbirths, are known to be more susceptible to microbial pathogenesis. Alteration of the fungi microenvironment, including changes in pH, temperature, osmotic pressure, and hormonal concentrations, is currently considered to be accountable for the initiation of C. albicans infection symptoms.

Women can contract vaginal candidiasis by engaging in sexual activity with men who may not be aware they are carrying the infection because the symptoms are so mild in men that they are either overlooked or are completely unfelt.

It has recently come to light that candidiasis strikes immunocompetent individuals as well. For example, the widespread use of anti-microbial agents, such as broad spectrum antibiotics, has resulted in a number of serious clinical consequences. For example, antibiotics can kill beneficial, non-pathogenic microorganisms (i.e., flora) within the gastrointestinal tract, but are powerless against the yeasts in the GI tract. As a result, the gastrointestinal yeasts normally kept in check by the flora begin to grow at an excessive rate. In addition to antibiotics, the unrestrained usage of steroids, birth control pills, antacid and anti-ulcer medications, as well as diets high in sugar have been blamed for the rapid rise in candidiasis in the general population. It is theorized that approximately 85% of Americans are or have been infected by some strain of yeast or fungus. Fungal infections can be systemic, subcutaneous, cutaneous or superficial (involving the outermost skin or hair).

Common candidiasis symptoms include, but are not limited to, fatigue or lethargy, depression, headaches, muscle aches, pain and/or swelling in the joints, irritability, memory loss, anxiety, and insomnia.

Fungal infections are among the most difficult to effectively treat and regimens must be continued for months before results can be seen. Currently, there are several different types of drugs on the market that can provide effective anti-fungal therapy, as well as anti-fungal diets, such as the four step Candida diet. The Candida diet includes: the immediate elimination of antibiotics, birth control pills and other hormone-altering substances; a low-sugar, low-carbohydrate, high protein diet; the medical use of anti-fungal agents such as nystatin; and the strengthening of the immune system through supplementation. Though often effective, anti-fungal drugs and the Candida diet are cost prohibitive to many and more often than not, the symptoms return upon termination of the regimen.

Manic depression or bipolar disorder is a neurological brain disorder involving extreme swings in mood, i.e., recycling between periods of mania and periods of depression. Manic depression is one of four mood disorders, the others being unipolar depressive disorder (depression only), unipolar disorder (mania only), and schizoaffective disorder (See Manual IV, American Psychiatric Association).

Currently, treating bipolar and unipolar disorders consists of a combination of psychotherapy, teaching learning life-adjustment skills, and using mood stabilizers such as lithium, and anticonvulsant medications such as valproic acid. The goal of the treatment is to effect a mental change in the person suffering from the disorder, from a state characterized by mood swings between mania and depression, to a stabilized state.

Lithium has a number of disadvantages including, but not limited to, the importance of establishing and maintaining the concentration of lithium in the blood, as well as a plethora or physiological conditions including hypothyroidism, tremors, dry mouth, weight gain, increased frequency of urination, nausea, impotence, decreased libido, diarrhea, kidney abnormalities, loss of appetite, visual impairment, seizures and arrhythmias. Valproic acid has been associated with hepatic dysfunction.

A highly desirable intervention would involve administration of a therapeutic agent that is safe and effective, without the attendant disadvantages of lithium and valproic acid to treat a patient with bipolar disorder for prevention or reduction of the biphasic mania/depression cycle and the stabilization of the patient in a mental state that is not subject to mood swings.

### SUMMARY OF THE INVENTION

The invention generally relates to the therapeutic treatment of bipolar disorder and associated disease states and physiological conditions, as well as therapeutic compositions and kits comprising same that are useful for such treatment.

In one aspect, the present invention relates to a method of treating a disease state including bipolar disorders comprising administering, to a subject in need thereof, an effective amount of a probiotic composition comprising (1) Bacillus subtilis, (2) Bacillus coagulans, and (3) Enterococcus faecium.

In another aspect, the present invention relates to a therapeutic probiotic composition having utility for oral administration for treatment or prophylaxis of a bipolar disorder disease state in a subject in need thereof. Such probiotic composition comprises (1) Bacillus subtilis, (2) Bacillus coagulans, and (3) Enterococcus faecium, and may optionally further comprise a non-microbial therapeutic agent effective for treatment or prophylaxis of bipolar disorder.

In a further aspect, the present invention relates to a kit for therapeutic intervention in treating or preventing a bipolar disorder disease state, such kit comprising:
(i) a probiotic composition comprising (1) Bacillus subtilis, (2) Bacillus coagulans, and (3) Enterococcus faecium; and
(ii) instructions for the administration of the probiotic composition to a subject in need of treatment or prophylaxis of the bipolar disorder disease state.

Other aspects, features and embodiments of the invention will be more fully apparent from the ensuing disclosure and appended claims.

### DETAILED DESCRIPTION OF THE INVENTION

The present invention generally relates to probiotic compositions and methods for treatment of disease states such as bipolar disorder. The compositions and methods of the invention may be employed for treating a patient suffering from such disease state or other related adverse physiological conditions, by administration of an effective dose of the probiotic composition to a patient in need of treatment thereof.

Applicants have unexpectedly discovered that the probiotic compositions of the present invention are able to effectively ameliorate and/or regulate bipolar disorders and many of the associated symptoms of such disorder.

The gastrointestinal microflora has been shown to play a number of vital roles in maintaining gastrointestinal tract function and overall physiological health. For example, the growth and metabolism of the many individual bacterial species inhabiting the gastrointestinal tract depend primarily upon the substrates available to them, most of which are derived from the diet. See e.g., Gibson G. R. et al., 1995. Gastroenterology 106: 975-982; Christi, S. U. et al., 1992. Gut 33: 1234-1238. These findings have led to attempts to modify the structure and metabolic activities of the community through diet, primarily with probiotics, which are live microbial food supplements. Probiotic microorganisms are those which confer a benefit when grown in a particular environment, often by inhibiting the growth of other biological organisms in the same environment. The best known probiotics are the lactic acid-producing bacteria (i.e., Lactobacilli) and Bifidobacteria, which are widely utilized in yogurts and other dairy products. These probiotic organisms are non-pathogenic and non-toxigenic, retain viability during storage, and survive passage through the stomach and small intestine. Since probiotics do not permanently colonize the host, they need to be ingested regularly for any health promoting properties to persist. Commercial probiotic preparations are generally comprised of mixtures of Lactobacilli and Bifidobacteria, although yeast such as Saccharomyces has also been utilized.

Although a potential link between bipolar disorder and gastrointestinal microflora has previously been a subject of speculation, no correlation has previously been conclusively scientifically established. Nonetheless, the applicants have found that administration of the probiotic compositions of the present invention are effective to modulate gastrointestinal microflora and assist in the maintenance of a stable mental state.

The probiotic bacterial composition of the present invention includes lactic-acid producing bacteria and soil bacteria. In such composition, the bacilli Bacillus subtilis, Enterococcus faecium, and Bacillus coagulans are utilized. Specifically, in the broad practice of the invention, the probiotic bacterial species in the composition of the present invention may comprise, consist of, or consist essentially of Bacillus subtilis, Enterococcus faecium, and Bacillus coagulans. In various embodiments, the bacterial species of the composition consist only of Bacillus subtilis, Enterococcus faecium, and Bacillus coagulans.

The microbial species in the probiotic composition of the present invention include Bacillus coagulans, which has the ability to form spores. The ability to sporulate makes such bacterial species relatively resistant to heat and other conditions, provides for a long shelf-life in product formulations, and is ideal for survival and colonization of tissues under conditions of pH, salinity, and the like within the gastrointestinal tract. Additional useful properties include being non-pathogenic, aerobic, facultative and heterotrophic, thus rendering such bacterial species safe and able to readily colonize the gastrointestinal tract.

It should be noted that Lactobacillus sporogenes has recently been re-characterized as Bacillus coagulans. The initial classification as Lactobacillus sporogenes (see, Nakamura et al., Int. J. Syst. Bacteriol., 38: 63-73, 1988) has been argued as incorrect due to the fact that the Lactobacillus sporogenes strain produces spores and through metabolic processes excretes L(+)-lactic acid, which under current classification rules requires that the bacterium be classified as a bacillus species. Furthermore, classic Lactobacillus species are normally unsuitable for colonization of the gut due to their instability in the harsh (i.e., acidic) pH environment of the bile, particularly human bile, but Lactobacillus sporogenes (Bacillus coagulans) is able to survive and colonize the gastrointestinal tract.

Bacillus coagulans is a non-pathogenic, Gram positive, spore-forming bacteria that produces L(+) lactic acid (dextrorotatory) under homo-fermentation conditions. It has been isolated from natural sources, such as heat-treated soil samples inoculated into nutrient medium (see e.g., Bergey's Manual of Systemic Bacteriology, Vol. 2, Sneath, P. H. A. et al., eds., Williams & Wilkins, Baltimore, Md., 1986). Bacillus coagulans has also been utilized to produce lactic acid (U.S. Pat. No. 5,079,164). Though not naturally found in the gut, Bacillus coagulans strains have been used as general nutritional supplements and agents to control constipation and diarrhea in humans and other animals.

Bacillus coagulans strains and their growth requirements have been described previously (see e.g., Baker, D. et al, Can. J. Microbiol., 6: 557-563, 1960; Nakamura, H. et al, Int. J. Syst. Bacteriol. 38: 63-73, 1988). In addition, various strains of Bacillus coagulans can be isolated from natural sources (e.g., heat-treated soil samples) using well-known procedures (see e.g., Bergey's Manual of Systemic Bacteriology, Vol. 2, p. 1117, Sneath, P. H. A. et al., eds., Williams & Wilkins, Baltimore, Md., 1986).

Bacillus subtilis is a Gram-positive, endospore-forming soil bacterium comprising aerobic and a few facultatively anaerobic rod-shaped bacteria. Bacillus subtilis was historically used to treat dysentery. It has been reported that ingested Bacillus subtilis is able to activate the human immune defense, including the IgM, IgG and IgA antibodies. Bacillus subtilis strains and their growth requirements are well known in the art.

Enterococcus faecium is a facultative anaerobic, Gram positive, cocci that produces L(+) lactic acid. The Enterococcus faecium strain is a natural inhabitant of the mammalian G.I. tract and causes many of the same problems as other members of the intestinal flora, including opportunistic urinary tract infections and wound infections.

The compositions of the invention comprising the bacilli (A) Bacillus subtilis, (B) Enterococcus faecium, and (C) Bacillus coagulans are suitably administered to a subject in need of treatment in an amount sufficient to elicit a therapeutically beneficial response or alternatively, to enhance or sustain an ongoing beneficial physiological response. The compositions of the invention can also be administered to a subject in need of treatment in an amount sufficient to effectively ameliorate and/or regulate symptoms associated with other disease states or physiological conditions.

The invention contemplates compositions comprising the bacilli Bacillus subtilis (“A”), Enterococcus faecium (“B”), and Bacillus coagulans (“C”), in which each of such bacilli A, B and C may vary in relative amount with respect to one another, in various embodiments of the invention. For example, each of A, B and C may be present in an equal or equivalent amount, in relation to one another. In other embodiments, A, B and C may be present in unequal amounts in relation to one another, with each of A, B and C being present in an amount in a range of from about 5% to about 90% by weight, based on the total weight of A, B and C, and with all amounts of A, B and C totaling 100%. Thus, the invention contemplates a wide variety of specific formulation embodiments, including by way of example the following illustrative compositions (showing percentages by weight for each of the A, B and C ingredients, based on the total weight of A+B+C in the composition):

| Weight percentages for | | | | Total wt |
|---|---|---|---|---|
| each of A, B, C | | | | % of A, |
| in Composition No: | A wt % | B wt % | C wt % | B and C |
| 1 | 30 | 30 | 40 | 100 |
| 2 | 25 | 40 | 35 | 100 |
| 3 | 25 | 50 | 25 | 100 |
| 4 | 25 | 70 | 5 | 100 |
| 5 | 20 | 40 | 40 | 100 |
| 6 | 20 | 50 | 30 | 100 |
| 7 | 20 | 60 | 20 | 100 |
| 8 | 10 | 50 | 40 | 100 |
| 9 | 10 | 60 | 30 | 100 |
| 10 | 10 | 80 | 10 | 100 |
| 11 | 5 | 80 | 15 | 100 |
| 12 | 5 | 90 | 5 | 100 |

In a specific embodiment, the probiotic composition of the invention comprises Enterococcus faecium, Bacillus subtilis and Bacillus coagulans as the only microbial species of the composition, i.e., the microbial species of the composition consist of Enterococcus faecium, Bacillus subtilis and Bacillus coagulans. The composition in addition to the microbial species may comprise any suitable ingredients that are pharmaceutically acceptable and that are not inconsistent with the beneficial character of the composition in combating bipolar disorder.

In one specific embodiment, the composition of the invention includes the Enterococcus faecium, Bacillus subtilis and Bacillus coagulans microbial ingredients, together with a support or substrate material for the microbial species, such as saccharides, oligosaccharides, and/or polysaccharides, or the like. The composition may for example include fructo-oligosaccharides (FOS) with the microbial species, packaged together in a dose form such as a capsule adapted for oral administration. Alternatively, the dose form can include powders, tablets, sachets, solutions, suspensions, elixers, or any other dose form that is efficacious for its intended purpose in combating bipolar disorder.

The specific relative amounts of the active ingredients in the compositions of the invention will depend on a variety of factors known to those of skill in the art of therapeutic formulation. For example, factors to be considered include the route of administration and the nature of the subject to be treated. The effect of such factors, and other factors known in the art such as synergistic effects, is readily determined by one of skill in the art according to standard clinical techniques. Effective doses of the active components of the compositions of the present invention may also be extrapolated from dose-response curves derived from animal model test systems. Therapeutic results accumulated to date suggest that the gender and age of the subject are irrelevant to the overall efficacy of the composition, with the exception of patients less than six years of age, who are generally more responsive to the composition than any other age group.

Although the disclosure herein is primarily directed to the treatment or prophylaxis of bipolar disorder, the compositions of the invention have also been found efficacious in the treatment or prophylaxis of other disease states and physiological conditions, including, for example, conditions such as deleterious fungal overgrowth, Candidiasis, autism (including autism per se as well as milder variants such as Aspergers syndrome), delayed development, acid reflux disease, vaginal yeast infections, impaired hearing, chronic ear infections, seasonal allergies, depression, Fibromyalgia syndrome, Crohn's disease, colitis, irritable bowel syndrome, interstitial cystitis, acne, sinusitis, rheumatoid arthritis, chronic fatigue syndrome, asthma, attention deficit disorder, attention deficit/hyperactivity disorder, rosacea, multiple sclerosis, hyperglycemia, Meniere's disease, Addison's disease, Parkinson's disease, Lou Gehrig's disease, ankylosing spondylitis, celiac disease, Graves' disease, Guillain-Barre' syndrome, Hashimoto's disease, lupus erythematosus, myasthenia gravis, Sjogren's syndrome, alopecia universalis, endometriosis, eczema, seborrheic dermatitis, jock itch, hives, athlete's foot, psoriasis, sarcoidosis, schizophrenia, scleroderma, ulcerative colitis and vulvodynia.

The composition of the invention may be administered in any suitable dose amount that is effective to prevent, ameliorate, regulate, cure or otherwise treat the disease state or physiological condition in a subject in need of such therapeutic intervention.

In various specific embodiments, an effective dose of the probiotic composition of the present invention is about 1.0 g to about 15.0 g for an adult patient, more preferably between about 2.0 g and about 10.0 g. Effective doses are to be administered to a patient in need at least once a week, preferably once a day. Pediatric dosages may be in the range of 15% to 90% of adult dosages.

For example, some patients may administer a constant dosage of the probiotic composition over time, for example about 2 g to about 4 g per day, while some patients may choose to increase the dosage up to about 6 g to about 10 g per day, depending on the severity of the disease state or physiological condition. Once the disease state or physiological condition has been effectively ameliorated or regulated, the patient may decrease the dosage to about 2 g to about 4 g per day for maintenance purposes. The patient may be a human patient, or a veterinary subject.

In general, the effective dosage of compositions of the invention for therapeutic use may be widely varied in the broad practice of the invention, depending on the specific application, subject, condition, or disease state involved, as readily determinable within the skill of the art. By way of illustration, in some embodiments of the invention, the effective dosage of the composition may be in a range of from about 10 milligrams (mg) to 200 milligrams (mg) per kilogram body weight of the recipient per day. The desired dose may be presented in multiple (e.g., two, three, four, five, six, or more) sub-doses administered at appropriate intervals throughout the day. These sub-doses may be administered in unit dosage forms containing an appropriate amount of active ingredients per unit dosage form.

The probiotic compositions of the invention may additionally and optionally comprise any suitable adjuvants, excipients, additives, carriers, additional therapeutic agents, bioavailability enhancers, side-effect suppressing components, or other ingredients that do not preclude the efficacy of the composition for ameliorating or regulating the disease states or physiological conditions. Preferably, the bacilli (Bacillus subtilis, Enterococcus faecium, and Bacillus coagulans) comprise from about 50% to about 90% by weight of the composition, based on the total weight of the anti-fungal composition of the invention. Most preferably, the bacilli comprise from about 60% to about 80% by weight of the composition.

The compositions of the present invention suitable for oral administration may be presented as discrete units such as gelatin capsules or sachets, each containing a predetermined amount of the bacterial composition in the form of a powder or granules.

Various routes of administration are contemplated other than oral administration, including, but not limited to, subcutaneous, intramuscular, intradermal, transdermal, intraocular, intraperitoneal, mucosal, vaginal, rectal, and intravenous administration routes.

In addition, the compositions of this invention may further include one or more accessory ingredients selected from diluents, buffers, flavoring agents, binders, preservatives, and the like.

The invention additionally contemplates the provision of therapeutic compositions, including the aforementioned microbial species in combination with other bipolar disorder treatment agents, e.g., lithium, anticonvulsant medications, antipsychotic medications, and combinations thereof, in additive as well as synergistic combinations of the microbial species and other therapeutic ingredients.

In another aspect, the invention relates to a kit including the probiotic compositions of the invention, with or without other bipolar disorder treatment agents, and written indicia for a corresponding therapeutic intervention, e.g., including a dosage regimen and related instructions.

The bacterial species may be in a dried form, e.g., lyophilized or sporolated form, in a suitable carrier medium, e.g., fructo-oligosaccharide (FOS) medium, or other soluble fiber, sugar, nutrient or base material for the composition, with which the bacterial species can be presented in an orally administrable form.

In one specific aspect, the probiotic composition comprises (i) Bacillus subtilis, (ii) Bacillus coagulans and (iii) Enterococcus faecium, in a physiologically compatible carrier medium, wherein the ratio of Bacillus subtilis:Bacillus coagulans:Enterococcus faecium is in a range of from 1:1.5:0.5 to 1:6:2.

In another specific aspect, the probiotic composition comprises the following bacterial components (i), (ii) and (iii):

(i) Bacillus subtilis, in an amount of 25 million to 1.5 billion colony forming units (CFUs)/1.5 gm dose
(ii) Bacillus coagulans in an amount of 50 million to 2 billion colony forming units (CFUs)/1.5 gm dose; and
(iii) Enterococcus faecium in an amount of 25 million to 1.5 billion colony forming units (CFUs)/1.5 gm dose,
and a carrier medium, e.g., wherein a 1.5 gm dose contains from 5% to 75% of such bacterial components (i), (ii) and (iii), and from 25% to 95% of the carrier medium, wherein the CFU ratio of Bacillus coagulans to Bacillus subtilis is in a range of from 1 to 6, inclusive, and wherein the CFU ratio of Enterococcus faecium to Bacillus subtilis is in a range of from 0.25 to 2, inclusive.

The composition in a further aspect includes a dose form comprising

(i) Bacillus subtilis, in an amount of 25 million to 1.5 billion colony forming units (CFUs);
(ii) Bacillus coagulans in an amount of 50 million to 2 billion colony forming units (CFUs); and
(iii) Enterococcus faecium in an amount of 25 million to 1.5 billion colony forming units (CFUs).

The composition in a still further aspect includes a dose form comprising

(i) Bacillus subtilis, in an amount of 100 million colony forming units (CFUs);
(ii) Bacillus coagulans in an amount of 200 million colony forming units (CFUs);
(iii) Enterococcus faecium in an amount of 100 million colony forming units (CFUs); and
(iv) fructo-oligosaccharides (FOS) in an amount of from 500 to 1000 mg.

Such dose form in a further specific aspect includes the bacterial species in a non-microencapsulated form.

Such dose form in a still further aspect comprises a capsule containing the composition, wherein the capsule is devoid of any enteric coating.

A further aspect of the disclosure relates to a probiotic composition in an oral dose form comprising, as the only bacterial species therein, the bacterial species of Bacillus subtilis, Bacillus coagulans, and Enterococcus faecium.

Another aspect of the disclosure relates to an oral dose form probiotic composition wherein the only bacterial species therein are:

(i) Bacillus subtilis, in an amount of 25 million to 1.5 billion colony forming units (CFUs);
(ii) Bacillus coagulans in an amount of 50 million to 2 billion colony forming units (CFUs); and
(iii) Enterococcus faecium in an amount of 25 million to 1.5 billion colony forming units (CFUs).

Yet another aspect of the invention relates to a method of augmenting a diet of a subject with a probiotic supplement, comprising administration to the subject of a composition of the present disclosure.

The invention in another aspect relates to a method of treatment, prevention, amelioration and/or regulation of a physiological condition susceptible to therapeutic intervention by probiotic administration, comprising administering to a subject afflicted with or susceptible to such physiological condition an effective amount of the composition of the present disclosure.

The physiological condition related to such method in one embodiment includes one or more of bipolar disorder, attention deficit disorder, attention deficit/hyperactivity disorder, and schizophrenia.

In another aspect, the disclosure relates to a method of combating bipolar disorder, comprising administering to a subject, e.g., a human subject, in need thereof, an effective oral dose of a probiotic composition including as the only bacterial species therein Bacillus subtilis, Bacillus coagulans, and Enterococcus faecium.

Such effective dose of the probiotic composition may contain the following bacterial species in the following amounts:

(i) Bacillus subtilis, in an amount of 25 million to 1.5 billion colony forming units (CFUs);
(ii) Bacillus coagulans in an amount of 50 million to 2 billion colony forming units (CFUs); and
(iii) Enterococcus faecium in an amount of 25 million to 1.5 billion colony forming units (CFUs).

In a further embodiment, the probiotic formulation of the disclosure comprises Bacillus coagulans (e.g., 200 million CFU), Bacillus subtilis (e.g., 100 million CFU), and Enterococcus faecium (e.g., 100 million CFU) as the only microbial species in the composition, in combination with fructo-oligosaccharides (FOS) and no other ingredients, packaged in a gelatin capsule dose form. Such composition may be used to effectively ameliorate the symptoms of bipolar disorder

The term “effectively ameliorate” refers to substantial improvement in the symptoms associated with the subject disease state or physiological condition, e.g., at least 50% improvement in such symptoms.

In the compositions of the invention, the bacilli Bacillus subtilis, Bacillus coagulans and Enterococcus faecium can be varied in relative therapeutic amounts with respect to one another. For example, in various specific embodiments, the ratio of Bacillus subtilis:Bacillus coagulans:Enterococcus faecium can be in a range of from 1:0.5:0.5 to 1:8:4. In other embodiments, each of the bacterial species is present in an amount of at least 25 million colony forming units (CFUs), e.g., a composition containing 25 million CFUs Bacillus subtilis, 200 million CFUs Bacillus coagulans and 25 million CFUs Enterococcus faecium.

In one specific aspect, the composition comprises (i) Bacillus subtilis, (ii) Bacillus coagulans and (iii) Enterococcus faecium, in a physiologically compatible carrier medium, wherein the ratio of Bacillus subtilis:Bacillus coagulans:Enterococcus faecium is in a range of from 1:1.5:0.5 to 1:6:2.

The physiologically compatible carrier medium with which the bacterial species are employed, can be of any simple type, e.g., a pharmaceutically acceptable carrier such as fructo-oligo-saccharide (FOS) medium, or other soluble fiber, sugar, nutrient or base material for the composition, with which the bacterial species can be formulated, e.g., in an orally administrable form. Other carrier media include mannitol, inulin (a polysaccharide), polydextrose, arabinogalactan, polyolslactulose, lactitol, etc. A wide variety of materials can be used as carrier material in the practice of the present disclosure, as will be apparent to those of ordinary skill in the art, based on the description herein.

The carrier medium, when present, can be blended with the bacterial species Bacillus subtilis, Bacillus coagulans and Enterococcus faecium in any suitable amounts, such as an amount of from 5% to 95% by weight of carrier medium, based on the total weight of the three bacterial species and the carrier medium, in various embodiments.

In other embodiments, the amount of carrier medium may be in a range having a lower limit of any of 5%, 10%, 12%, 15%, 20%, 25%, 28%, 30%, 40%, 50%, 60%, 70% or 75%, and an upper limit, higher than the lower limit, of any of 20%, 22%, 25%, 28%, 30%, 40%, 50%, 60%, 70%, 75%, 80%, 85%, 90%, and 95%.

The amount of carrier medium in a specific embodiment may be determined based on considerations of the specific dose form, relative amounts of the three bacterial species, the total weight of the composition including the carrier medium and the bacterial species, and the physical and chemical properties of the carrier medium, and other factors, as known to those of ordinary skill in the probiotic formulation art.

In various specific embodiments, the composition comprises:

(i) Bacillus subtilis, in an amount of 25 million to 1.5 billion colony forming units (CFUs)/1.5 gm dose
(ii) Bacillus coagulans in an amount of 50 million to 2 billion colony forming units (CFUs)/1.5 gm dose; and
(iii) Enterococcus faecium in an amount of 25 million to 1.5 billion colony forming units (CFUs)/1.5 gm dose,
and a carrier medium.

Such 1.5 gm dose can for example contain from 5% to 75% of such bacterial components (i), (ii) and (iii), and from 25% to 95% of the carrier medium. It will be recognized that such 1.5 gm dose has been set forth above as one illustrative dosage of the composition of the disclosure, with respect to which the amounts of the various components of the composition have been specified.

In other embodiments of the disclosure, compositions are provided wherein the CFU ratio of Bacillus coagulans to Bacillus subtilis is in a range of from 1 to 6, inclusive, and wherein the CFU ratio of Enterococcus faecium to Bacillus subtilis is in a range of from 0.25 to 2, inclusive. The probiotic compositions of the disclosure can be formulated in any suitable manner, e.g., by culturing of the respective bacterial components, and by simple mixing thereof with the other ingredients, and the compositions in various embodiments are formulated to contain only the bacterial species identified herein.

The composition in a further embodiment includes a dose form comprising

(i) Bacillus subtilis, in an amount of 25 million to 1.5 billion colony forming units (CFUs);
(ii) Bacillus coagulans in an amount of 50 million to 2 billion colony forming units (CFUs); and
(iii) Enterococcus faecium in an amount of 25 million to 1.5 billion colony forming units (CFUs).

The composition in another embodiment constitutes a dose form comprising

(i) Bacillus subtilis, in an amount of 100 million colony forming units (CFUs);
(ii) Bacillus coagulans in an amount of 200 million colony forming units (CFUs);
(iii) Enterococcus faecium in an amount of 100 million colony forming units (CFUs); and
(iv) fructo-oligosaccharides (FOS) in an amount of from 500 to 1000 mg.

The compositions of the disclosure, including the bacterial species and optionally a suitable carrier medium, can be provided in any suitable dose form for administration. The dose form is preferably a pill, powder, capsule, sachet, or the like, formulated for oral administration.

One preferred embodiment of the dose form is a capsule containing the composition of the disclosure including the three bacterial species in a dried form, blended with FOS. The composition may for example be constituted by 100 million CFUs of Bacillus subtilis, 200 million CFUs of Bacillus coagulans, and 100 million CFUs of Enterococcus faecium, with 660 mg of FOS in a 750 mg capsule.

The capsule for such dose form can be of any suitable type, e.g., a gelatin capsule of a conventional variety. Preferably, such capsule is devoid of any enteric coating. Preferably the bacterial species are non-microencapsulated in the dose form.

It will be appreciated that the composition of the disclosure can be usefully employed for combating a variety of adverse physiological conditions for which probiotic treatment is advantageous. It will be further appreciated that the probiotic composition of the disclosure can be usefully employed as a food additive in foodstuffs of widely varied types, to realize the health, dietary and nutritional benefits of such composition.

The dosing regimen involving compositions in accordance with the present disclosure can be varied to achieve a desired result, such as may be determined empirically for a given individual subject, or by extrapolation from data obtained from administering the composition to a clinical or other test population. For example, the dosage regimen may be carried out to administer each of the three bacilli Bacillus subtilis, Bacillus coagulans and Enterococcus faecium, in an amount that is within a range of from 40 million CFUs up to 2 billion CFUs per day for each of such bacilli, and wherein the relative amounts of each may be the same as or different from the others, each being independently selected from such range. The dose forms may be constituted as necessary for a specific dosing regimen, and such regimen may maintained or varied as appropriate to the desired result.

In general, the composition of the disclosure may be administered in any suitable dose amount that is effective as a health supplement, food supplement, food additive, and/or therapeutic agent to prevent, ameliorate, regulate, cure or otherwise treat a disease state or physiological condition in a subject in need of such intervention. As used herein, the phrase “effective amount” means an amount including at least 10 million CFUs of Bacillus subtilis, at least 100 million CFUs of Bacillus coagulans and at least 10 million CFUs of Enterococcus faecium.

In various specific embodiments, an effective dose of a composition of the present disclosure can be in a range of from 1.0 gm to 15.0 gm for an adult patient, more preferably between about 2.0 gm and about 10.0 gm of the composition. Effective doses can be administered to a subject at any suitable frequency, e.g., at least once a week, preferably once a day. Pediatric dosages may be in the range of 15% to 90% of adult dosages.

In therapeutic applications for combating an adverse physiological condition, a constant dosage of the composition can be administered over time, for example about 2 gm to about 4 gm per day, up to about 6 g to about 10 g per day, depending on the severity of the physiological condition. Once the physiological condition has been effectively ameliorated, the subject can in many instances decrease the dosage to about 2 gm to about 4 gm per day for maintenance purposes.

The desired dose may be presented in multiple (e.g., two, three, four, five, six, or more) sub-doses administered at appropriate intervals throughout the day.

The compositions of the disclosure may additionally and optionally include any suitable adjuvants, excipients, additives, carriers, additional therapeutic agents, bioavailability enhancers, side-effect suppressing components, diluents, buffers, flavoring agents, binders, preservatives or other ingredients that do not preclude the efficacy of the composition. In various preferred embodiments, the bacilli (Bacillus subtilis, Enterococcus faecium, and Bacillus coagulans) comprise from about 50% to about 90% by weight of the composition, based on the total weight of the composition including a carrier medium, most preferably from about 60% to about 80% by weight of the composition. It is generally preferred that the composition contain only the bacilli and a carrier medium, in a gelatin capsule that is devoid of any enteric coating, with the bacteria being in a dried form such as powder or granules, and the carrier medium likewise being in a dry powder or particulate form.

The compositions of the present disclosure are preferably formulated for oral administration. Other routes of administration can be employed, however, including, but not limited to, subcutaneous, intramuscular, intradermal, transdermal, intraocular, intraperitoneal, mucosal, vaginal, rectal, and intravenous. For example, a dose of the composition of the present disclosure can be presented in a gelatin capsule and inserted vaginally to treat vaginal yeast infections.

In one embodiment, the therapeutic composition of the disclosure may comprise at least 40 million CFUs of each of Bacillus subtilis, Bacillus coagulans and Enterococcus faecium, as contained in capsules containing from 25 to 200 milligrams of the mixture Bacillus subtilis, Bacillus coagulans and Enterococcus faecium with one or more excipients, such as 500-1,000 milligrams of fructo-oligosaccharides, in a unitary dose form tablet or capsule.

In one advantageous form, the probiotic composition contains Bacillus coagulans (200 million CFU), Bacillus subtilis (100 million CFU), Enterococcus faecium (100 million CFU) as the only microbial species in the composition, in combination with fructo-oligosaccharides (FOS) and no other ingredients, as packaged in a gelatin capsule dose form. Such composition is referred to hereinafter as the “Symbion formulation.”

The Symbion formulation may be administered and taken orally according to any effective dosing regimen. In one embodiment, the beginning dose is one capsule taken after a meal. After one week, the dose is increased to one capsule twice daily. At four weeks, the dose is increased to two capsules, twice daily. Up to six capsules a day may be taken, but the recommended long term dose is four capsules per day.

The compositions of the disclosure are readily manufactured. The respective bacterial species Bacillus subtilis, Bacillus coagulans and Enterococcus faecium are seeded from standard stock into a reactor and grown in standardized media until a predetermined CFU/gm concentration is reached. The bulk material then is drained from the reactor and dried by spray drying, lyophilization, or flatbed oven drying. The dried bacterial raw material thereafter is blended with the carrier medium and the resulting mixture can be pressed into tablets, filled into foil pouches as a granular solid, or introduced into gelatin capsules as a particulate material.

The bacterial species therefore are present in the dose form as live bacteria, whether in dried, lyophilized, or sporolated form. Bacillus coagulans may for example be in a sporolated form, while Enterococcus faecium may be in a non-sporolated form.

By way of specific illustration, the alternative composition containing (i) Bacillus subtilis (ii) Bacillus coagulans and (iii) Enterococcus faecalis as the only microbial components of the composition can be formulated with 25 million CFUs of Bacillus subtilis, 200 million CFUs of Bacillus coagulans and 25 million CFUs of Enterococcus faecalis, and 90 wt. % of fructo-oligosaccharides (FOS).

The probiotic composition for such purpose advantageously is in an oral dose form comprising, as the only bacterial species therein, the bacterial species of Bacillus subtilis, Bacillus coagulans, and Enterococcus faecium. Such bacterial species may be present in the probiotic composition in such oral dose form in the following amounts: (i) Bacillus subtilis, in an amount of 25 million to 1.5 billion colony forming units (CFUs); (ii) Bacillus coagulans in an amount of 50 million to 2 billion colony forming units (CFUs); and (iii) Enterococcus faecium in an amount of 25 million to 1.5 billion colony forming units (CFUs). The probiotic composition may comprise fructo-oligosaccharide in the dose form, e.g., in an amount of from 500 to 1000 milligrams. The oral dose form may comprise a capsule containing the probiotic composition.

In various specific embodiments, the above-describe probiotic composition may contain the bacterial species in a ratio of Bacillus subtilis:Bacillus coagulans:Enterococcus faecium that is in a range of from 1:0.5:0.5 to 1:8:4, or in other particular embodiments, in a ratio of Bacillus subtilis:Bacillus coagulans:Enterococcus faecium that is in a range of from 1:1.5:0.5 to 1:6:2.

The disclosure therefore contemplates a method of combating bipolar disorder, comprising administering to a human subject in need thereof, an effective oral dose of a probiotic composition as variously described above. When in an oral dose form comprising a capsule containing (i) Bacillus subtilis, in an amount of 25 million to 1.5 billion colony forming units (CFUs); (ii) Bacillus coagulans in an amount of 50 million to 2 billion colony forming units (CFUs); and (iii) Enterococcus faecium in an amount of 25 million to 1.5 billion colony forming units (CFUs), and fructo-oligosaccharide in an amount of from 500 to 1000 milligrams, the oral dose form may be administered in a daily amount of from 1 capsule to 6 capsules. In specific embodiments, the oral dose form may be administered in any other suitable daily amounts, e.g., a daily amount of from 3 to 5 capsules.

In a specific implementation, the oral dose form can be administered daily in an amount, and for sufficient duration, to cause the elimination of bipolar disorder in a human subject. The duration of such treatment may for example be on the order of from 8 to 12 months.

The features and advantages of the disclosure are more fully shown by the following illustrative and non-limiting examples.

### Example 1

A fifty-six year-old female patient with bipolar disorder reported experiencing suicidal tendencies and being unable to focus enough to read or solve problems. The patient's existing medication included large doses of anti-psychotic drugs. The patient orally administered approximately 2 g of the anti-fungal composition of the present invention per day with water. After three months, the patient's daily dosage of the probiotic composition was up to about 6 g per day and she was able to read and solve problems while simultaneously reducing the intake of anti-psychotic medications by half, and the patient reported that her suicidal tendencies had been reduced by the probiotic medication.

### Example 2

A 56 year-old male patient with bipolar disorder was taking anti-anxiety and anti-depressants to maintain balance. The patient orally administered approximately 2 g of the probiotic composition of the present invention per day with water. After three months, the patient's daily dosage of the probiotic composition was up to about 6 g per day. The patient simultaneously eliminated sugar, bleached flour and gluten products from his diet, and was able to discontinue taking previously prescribed anti-anxiety and anti-depressant pharmaceuticals. Additionally, the patient reported the loss of feelings of hopelessness that had formerly plagued him.

While the invention has been described herein with reference to various specific embodiments, it will be appreciated that the invention is not thus limited, and extends to and encompasses various other modifications and embodiments, as will be appreciated by those ordinarily skilled in the art. Accordingly, the invention as hereinafter claimed is intended to be broadly construed and interpreted, as encompassing all such modifications and embodiments within the scope of the corresponding claims.

What is claimed is:

1. A probiotic composition comprising Bacillus subtilis, Enterococcus faecium, and Bacillus coagulans.

2. The probiotic composition of claim 1, wherein Bacillus subtilis, Enterococcus faecium, and Bacillus coagulans are the only microbial species in the composition.

3. The probiotic composition of claim 1, further comprising a substrate material for the microbial species in the composition.

4. The probiotic composition of claim 3, wherein the substrate material comprises material selected from among saccharides, oligosaccharides, and/or polysaccharides.

5. The probiotic composition of claim 3, wherein the substrate material comprises fructo-oligosaccharides.

6. The probiotic composition of claim 5, packaged in a dose form adapted for oral administration.

7. The probiotic composition of claim 6, wherein the dose form comprises a capsule.

8. The probiotic composition of claim 1, wherein the ratio of Bacillus subtilis:Bacillus coagulans:Enterococcus faecium is in a range of from 1:1.5:0.5 to 1:6:2.

9. The probiotic composition of claim 1, wherein
(i) Bacillus subtilis is in an amount of 25 million to 1.5 billion colony forming units (CFUs);
(ii) Bacillus coagulans is in an amount of 50 million to 2 billion colony forming units (CFUs);
(iii) Enterococcus faecium is in an amount of 25 million to 1.5 billion colony forming units (CFUs), and
(iv) fructo-oligosaccharides (FOS) in an amount of from 500 to 1000 mg.

10. The probiotic composition of claim 1, wherein the ratio of Bacillus subtilis:Bacillus coagulans:Enterococcus faecium can be in a range of from 1:0.5:0.5 to 1:8:4.

11. The probiotic composition of claim 1, wherein the CFU ratio of Bacillus coagulans to Bacillus subtilis is in a range of from 1 to 6, inclusive, and wherein the CFU ratio of Enterococcus faecium to Bacillus subtilis is in a range of from 0.25 to 2, inclusive.

12. The probiotic composition of claim 1, comprising at least 10 million CFUs of Bacillus subtilis, at least 100 million CFUs of Bacillus coagulans and at least 10 million CFUs of Enterococcus faecium.

13. The probiotic composition of claim 1, further comprising at least one ingredient selected from the group consisting of adjuvants, excipients, additives, carriers, additional therapeutic agents, bioavailability enhancers, side-effect suppressing components, diluents, buffers, flavoring agents, binders, and preservatives.

14. The probiotic composition of claim 1, wherein Bacillus subtilis, Enterococcus faecium, and Bacillus coagulans) comprise from about 50% to about 90% by weight of the composition, based on the total weight of the composition including a carrier medium.

15. The probiotic composition of claim 1, wherein Bacillus subtilis, Enterococcus faecium, and Bacillus coagulans) comprise from about 60% to about 80% by weight of the composition, based on the total weight of the composition including a carrier medium.

16. The probiotic composition of claim 1, comprising at least 40 million CFUs of each of Bacillus subtilis, Bacillus coagulans and Enterococcus faecium.

17. The probiotic composition of claim 1, wherein at least one of the microbial species is in a dried or lyophilized form.

18. The probiotic composition of claim 1, wherein at least one of the microbial species is in a sporolated form.

19. The probiotic composition of claim 1, further comprising a non-microbial therapeutic agent that is effective for the treatment of bipolar disorder.

20. A probiotic composition, wherein microbial species include (i) Bacillus subtilis, in an amount of 25 million to 1.5 billion colony forming units (CFUs); (ii) Bacillus coagulans in an amount of 50 million to 2 billion colony forming units (CFUs); and (iii) Enterococcus faecium in an amount of 25 million to 1.5 billion colony forming units (CFUs), and further comprising fructo-oligosaccharide in an amount of from 500 to 1000 milligrams.
